(19) United States
(12) Patent Application Publication
(10) Pub. No.: US 2021/0161957 A1
(43) Pub. Date: Jun. 3, 2021

(54) NK CELLS AND ANTIBODIES FOR CANCER TREATMENT

(71) Applicant: University Health Network, Toronto (CA)

(72) Inventors: Armand KEATING, Toronto (CA); Brent Allen WILLIAMS, Markham (CA); Xing-Hua WANG, Toronto (CA)

(21) Appl. No.: 16/931,332

(22) Filed: Jul. 16, 2020

Related U.S. Application Data
(63) Continuation of application No. 15/571,593, filed on Nov. 3, 2017, now abandoned.

(30) Foreign Application Priority Data
May 5, 2015 (EP) 15166415.8
May 15, 2015 (JP) 2015-099732

(51) Int. Cl. A61K 35/17; C07K 16/28; A61P 35/02; A61K 39/395; C12N 5/0783

(52) U.S. Cl. CPC A61K 35/17; C07K 16/2803; A61K 2039/505; A61K 39/395; C12N 5/0646; A61P 35/02

(57) ABSTRACT

Treatment of cancer in a patient comprises administering to the patient an effective amount of an antibody and an effective amount of NK cells, wherein the antibody binds an antigen on the surface of the NK cells and the antibody binds to an Fc receptor on a cell of the cancer. Anticancer activity is via resultant killing action of the NK cell on the cancer cell now binding the antibody via R-ADCC.

### CROSS-REFERENCE TO RELATED APPLICATIONS AND INCORPORATION BY REFERENCE

This application is a continuation of U.S. application Ser. No. 15/571,593, filed Nov. 3, 2017, which is a national stage of PCT/CA2015/051223, filed Nov. 24, 2015, which claims priority to EP Application Number 15166415.8, filed May 5, 2015 and JP Application Number 2015-099732, filed May 5, 2015, the entire contents of each are incorporated by reference herein.

### INTRODUCTION

The present invention relates to NK cells, antibodies and to related methods, uses and compositions for treatment of cancers.

### BACKGROUND TO THE INVENTION

Acute myeloid leukemia (AML) is a hematopoietic malignancy involving precursor cells committed to myeloid development, and accounts for a significant proportion of acute leukemias in both adults (90%) and children (15-20%) (Hurwitz, Mounce et al. 1995; Lowenberg, Downing et al. 1999). Despite 80% of patients achieving remission with standard chemotherapy (Hurwitz, Mounce et al. 1995; Ribeiro, Razzouk et al. 2005), survival remains unsatisfactory because of high relapse rates from minimal residual disease (MRD). The five-year survival is age-dependent; 60% in children (Rubnitz 2012), 40% in adults under 65 (Lowenberg, Downing et al. 1999) and 10% in adults over 65 (Ferrara and Schiffer 2013). These outcomes can be improved if patients have a matched hematopoietic cell donor, but most do not, highlighting the need for an alternative approach to treatment.

Natural killer (NK) cells are cytotoxic lymphocytes, with distinct phenotypes and effector functions that differ from e.g. natural killer T (NK-T) cells. For example, while NK-T cells express both CD3 and T cell antigen receptors (TCRs), NK cells do not. NK cells are often found to express the markers CD16 and CD56, wherein CD16 functions as an Fc receptor and mediates antibody dependent cell-mediated cytotoxicity (ADCC) which is discussed below. Despite NK cells being naturally cytotoxic, NK cell lines with increased cytotoxicity have been developed. NK-92 and KHYG-1 represent two cell lines that have been researched extensively and show promise in cancer therapeutics (Swift et al. 2011; Swift et al. 2012).

KHYG-1 cells are known to be pre-activated. Unlike endogenous NK cells, KHYG-1 cells are polarized at all times, increasing their cytotoxicity and making them quicker to respond to external stimuli. NK-92 cells have a higher baseline cytotoxicity than KHYG-1 cells.

Furthermore, NK cells express both activating and inhibitory receptors on their surface. Upon binding of ligand to activating receptors, e.g. NKp30, signals are produced that give rise to a more cytotoxic NK phenotype. NKp30-mediated NK activation has been shown to result in increased killing of blood cancer cells (Muller et al. 2014). Moreover, WO 2005/009465 describes co-administration of NK cells with antibodies, specific for activating receptors on the NK cell surface, as a treatment for viral infections via ADCC.

In haplotype transplantation, the graft-versus-leukemia effect is believed to be mediated by NK cells when there is a KIR receptor-ligand mismatch, which can lead to improved survival in the treatment of AML (Ruggeri, Capanni et al. 2002; Ruggeri, Mancusi et al. 2005). Further, rapid NK recovery is associated with better outcome and a stronger GVL effect in patients undergoing haplotype T-depleted hematopoietic cell transplantation (HCT) in AML (Savani, Mielke et al. 2007). Other trials have used haploidentical NK cells expanded ex vivo to treat AML in adults (Miller, Soignier et al. 2005) and children (Rubnitz, Inaba et al. 2010).

Several permanent NK cell lines have been established, and the most notable is NK-92 (mentioned above), derived from a patient with non-Hodgkin's lymphoma expressing typical NK cell markers except for CD16 (Fc gamma receptor). NK-92 has undergone extensive preclinical testing and exhibits superior lysis against a broad range of tumours compared with activated NK cells and lymphokine-activated killer (LAK) cells (Gong, Maki et al. 1994). Cytotoxicity of NK-92 cells against primary AML has been established (Yan, Steinherz et al. 1998). Another NK cell line, KHYG-1, has been identified as a potential contender for clinical use (Suck et al. 2005) and retains its cytotoxicity when irradiated (Suck et al. 2006) but nevertheless has reduced cytotoxicity so has received less attention than NK-92, which have been shown to preferentially target AML stem cells (Williams et al. 2010).

ADCC is a well-known phenomenon in which NK cells recognize the Fc region of antibodies bound to target cells and promote target cell killing (Grier et al. 2012; Deng et al. 2014; Kobayashi et al. 2014). In order for this to occur, the NK cells must express Fc receptors (FcRs). Following binding of the NK FcRs to the Fc region of the antibodies, a more cytotoxic NK phenotype prevails. This often leads to increased killing of the target cells to which the antibody specifically binds.

Notter et al. demonstrated that precoating lymphokine activated killer (LAK) cells with anti-CD3 antibodies could enhance killing of autologous AML blasts. A combination of IL-2, IFN-γ and anti-CD3 monoclonal antibody was proposed as a potential treatment for AML.

However, all current adoptive immunotherapy protocols are affected by donor variability in the quantity and quality of effector cells, variables that could be eliminated if effective cell lines were available to provide more standardized therapy. T cells targeted to tumors is proposed as a therapy option, e.g. via chimeric antigen receptor (CAR) modifications, but this requires genetic manipulation of T cells.

There thus exists a need for alternative and preferably improved treatments for leukemias, including AML, other blood cancers in general and still more generally other cancers of humans.

An object of the invention is to provide alternative methods, uses and compositions for treatment of tumours, especially cancers, especially in humans. Embodiments have as object the provision of improved methods, uses and compositions. More particular embodiments aim to provide treatments for identified cancers, e.g. blood cancers such as leukemias. Specific embodiments aim to take advantage of combinations of antibodies and effector cells in cancer therapies.

### SUMMARY OF THE INVENTION

There are provided herein methods of treating cancer, using antibodies that bind to antigens on NK cells, wherein the combined complex of the antibodies with the NK cells then also binds to cancer cells. Also provided are the antibodies for use in such methods. The dogma in this field is that NK cells can have anti-tumourigenic properties that are antibody dependent, operating via antibody dependent cell-mediated cytotoxicity (ADCC). In this invention, surprisingly, while antibodies contribute to the NK cell-mediated killing of target cells they do so via a different mechanism.

According to the invention, there are provided methods of treating tumours, e.g. cancer, using a combination of the antibodies and NK cells. In examples, KHYG-1 cells, a CD16 negative NK population, are specifically used. Also provided is the combination of the antibody and the NK cell for use in such methods.

There are provided methods of treating tumours, e.g. cancer, using KHYG-1 type cells or a modified variant thereof. Again, these cells are also provided for use in such methods.

Compositions are provided comprising (a) a NK cell, and (b) an antibody that binds to a particular activating receptor on the NK cell. These are suitable for use in treatment of tumours and cancers.

Diseases particularly treatable according to the invention include cancers, blood cancers, leukemias, specifically acute myeloid leukemia. Tumours and cancers in humans in particular can be treated. References to tumours herein include references to neoplasms.

### DETAILS OF THE INVENTION

As described in detail below in examples, treatment of tumour cells, specifically cancer cells, has been achieved using antibodies and NK cells in combination. The inventors have shown utility of embodiments of the invention based on reverse antibody dependent cell-mediated cytotoxicity in vitro and using in vivo models of human cancer therapy.

According to the present invention there is therefore provided a method of treating a tumour in a patient, comprising administering an effective amount of an antibody to the patient, wherein the antibody binds an antigen on the surface of a natural killer (NK) cell and the antibody binds to an Fc receptor on a cell of the tumour.

In examples below, it is shown how the antibody may suitably bind an Fc receptor on the surface of the tumour cell, e.g. selected from CD16 (FcγRIII), CD32 (FcγII) or CD64 (FcγI). The antibody comprises an Fc region or otherwise a portion that is capable of binding an Fc receptor on a tumour cell. Ball et al. conducted a study of the expression of Fcγ receptors on primary AML and noted the following frequencies: FcγRI (58%); FcγII, (67%); and Fcγ III, (26%). FcγI and II receptor expression was highly correlated with FAB M4 and M5 morphology. Hence, treatments of the invention are suitable for tumours/cancers identified to express one or more Fc receptor. In one example, a humanized anti-NK cell activating receptor antibody alone is administered. Preferably, this antibody is anti-NKp30 or anti-NKp44.

As a result, binding of the antibody to the NK cell and binding of that bound combination to a tumour cell leads to tumour cell death. Hence, a role of the antibody is in effect to cross-link the effector NK cell to the target cancer cell. Typically, the antibodies comprise an Fc region (which by definition binds Fc receptors). Other suitable antibodies may comprise portions that are not strictly speaking Fc regions (for whatever reason) but which nevertheless in use bind the Fc receptors on the tumour cells rather than NK cells. It is thus a feature of particular embodiments of the invention that the NK cell does not express an Fc receptor.

Antibodies may be used as a set or plurality of antibodies having substantially all the same binding properties. Alternatively, mixtures may be used of antibodies that bind to different NK cell surface markers or proteins or of antibodies containing different Fc regions, or in fact different antibodies may differ in both respects within a plurality of antibodies to be used in the treatment.

Killing of tumour cells is achieved in use of specific embodiments of the invention through a mechanism referred to as reverse antibody dependent cell-mediated cytotoxicity (R-ADCC). Specific examples included herein demonstrate this for the first time supported by control and verification examples that now plausibly show the tumour cell cytotoxicity as a result of R-ADCC.

Methods, uses and compositions herein described, above and below, are suitable for treatment of cancer, in particular cancer in humans, e.g. for treatment of cancers of blood cells or solid cancers.

In preferred embodiments of the invention, killing of cancer cells is achieved by killing cancer stem cells, offering improved cancer therapy. Specific examples of the invention, set out below in more detail, demonstrate killing of clonogenic leukemia cancer cells.

Embodiments of the invention are especially suitable for treatment of hematologic cancers, being cancers of the blood, bone marrow and/or lymph nodes. These include leukemias, lymphomas and myelomas. Specific cancers treatable are selected from acute lymphocytic leukemia (ALL), acute myeloid leukemia (AML), chronic lymphocytic leukemia (CLL) and chronic myeloid leukemia (CML), Hodgkin's lymphoma, non-Hodgkin's lymphomas, including T-cell lymphomas and B-cell lymphomas, asymptomatic myeloma, smoldering multiple myeloma (SMM), active myeloma and light chain myeloma. In particular, the invention is of use to treat ALL, AML and B-cell lymphomas.

Further embodiments of the invention are especially suitable for treatment of cancers characterized by expression of Fc receptors on the cancer cell surface. These include ALL, AML and B-cell lymphomas and also melanoma, e.g. malignant melanoma.

Further specific examples of tumours treatable by the invention are selected from bladder carcinoma, chondrosarcoma, colorectal cancer, gastrointestinal cancer, glioma, head and neck cancer, kidney cancer, liver cancer, ovarian cancer, pancreatic cancer, soft tissue/muscle tissue cancer, prostate cancer, breast (mammary gland) cancer, germ cell cancer, multiple myeloma, histiocytic sarcoma, melanoma, skin cancer, uterine cancer and colon cancer.

Various routes of administration will be known to the skilled person to deliver active agents and combinations thereof to a patient in need. Embodiments of the invention are for blood cancer treatment. Administration of the antibody, or NK cells or combination thereof can be systemic or localized, such as for example via the intraperitoneal route

Increased targeting of the active agent is suitably achieved by means designed to home the agent to the tumour cells. NK cells may not be found in large numbers in advanced tumours. This can be a result of tumours interfering with cytokine/chemokine signalling. Intratumoral cytokine/chemokine therapy can be used target NK cells to cancers, e.g. IL-2, IL-12, IL-15 and IL-21 are all capable of activating NK cells at the tumour site and increasing lysis of the cancer cells (Zamai et al, 2007). Increased homing of NK effector cells to tumour sites may also be made possible by disruption of the tumour vasculature, e.g. by metronomic chemotherapy, or by using drugs targeting angiogenesis (Melero et al, 2014) to normalize NK cell infiltration via cancer blood vessels.

In other embodiments, active agent is administered more directly. Thus administration can be directly intratumoural, suitable especially for solid tumours. Administration can alternatively be intraperitoneal, such as in the case of metastatic ovarian cancer

Antibodies for use in the invention, whether alone or in combination with cells, preferably bind to cell surface antigens, proteins or markers on NK cells. Binding can attach the antibody leaving exposed, and separately available for binding, a portion that binds in turn to the tumour cells, via an Fc receptor. In examples, carried out and described herein the antibody binds to an antigen/receptor on the NK surface. Suitable receptors are known NK cell activating receptors, e.g. natural cytotoxicity receptors. In use below, the antibodies activated or otherwise turned on (were agonists for) these activating receptors. As individual examples are NKp30, NKp44, NKp46, CS-1 and NKG2D, the first two having been used successfully in demonstrating the effectiveness of the invention.

In an embodiment of the invention, NK cells are used in combination with an antibody specific for SLAMF7 (CS-1), to treat multiple myeloma (MM). The antigen is present on the NK cells, and hence treatment occurs via R-ADCC. KHYG-1 cells are preferably used and SLAMF7 antibodies are commercially available.

In another embodiment of the invention, NK cells are used in combination with an antibody, to treat AML. Known AML cells lack expression of SLAMF7 but do express CD32 (an Fc receptor). Therefore, ADCC using a SLAMF7 antibody is not possible, and treatment of AML occurs via R-ADCC. KHYG-1 cells are preferably used and SLAMF7 antibodies are commercially available.

In further embodiments, blocking antibodies are used, wherein the antibody binds and neutralizes an inhibitory receptor on the NK surface. Suitable antigens/receptors for the blocking antibodies include CD85d (LIR-2), CD85J (LIR-1), CD96 (TACTILE), CD152 (CTLA4), CD159a (NKG2A), CD223 (LAG-3), CD279 (PD-1), CD328 (SIGLEC7), SIGLEC9, TIGIT and TIM-3.

It is therefore preferred that the antibody is specific for an antigen expressed only on the surface of NK cells—the antigen is preferably not expressed on the cancer and preferably not expressed to a significant extent on any other cell. Nevertheless, if the NK cells are pre-coated with antibody, prior to administration, this level of specificity is less important.

The invention provides a combination therapy, hence a method described and claimed herein for treating a tumour in a patient comprises administering to the patient an effective amount of the antibody and an effective amount of NK cells, wherein:
the antibody binds an antigen on the surface of the NK cells; and
the antibody binds to an Fc receptor on a cell of the tumour.

Administration of the NK cells is in combination with the antibody, so an effect of the two together is realized, and can occur prior to, simultaneously with or subsequent to administering the antibody. In an example, exogenous NK cells, preferably KHYG-1 cells, are administered in combination with a humanized anti-NK activating receptor antibody, preferably anti-NKp30, the NK cells having had their proliferation capacity diminished, e.g. by irradiation.

The NK cell line KHYG-1 is preferable over other NK cell lines, e.g. NK-92, because the high baseline cytotoxicity of NK-92 cells may increase the risk of adverse effects upon administration to a patient, since normal cells of the body are less capable of dampening their cytotoxicity.

In carrying out the invention, treatment has been successful in which the antibody is administered bound to the NK cell. Pre-incubating the antibody with the NK cell prior to administering the antibody bound to the NK cell is one way to prepare this combination. In yet another example, the NK cells, such as KHYG-1, are pre-treated with a humanized anti-NK activating receptor antibody, preferably anti-NKp30, before administration, the NK cells having had their proliferation capacity diminished, e.g. by irradiation.

NK cells in general are believed suitable for the methods, uses and compositions of the invention. Exogenous NK cells are preferably used, though in embodiments a patient's own NK cells are used (e.g. ex vivo). As per cells used in certain examples herein, the NK cell can be a NK cell obtained from a cancer cell line. Advantageously, a NK cell, preferably treated to reduce its tumourigenicity, for example by rendering it mortal and/or incapable of dividing, can be obtained from a blood cancer cell line and used in methods of the invention to treat blood cancer. Antibodies are utilized that bind the NK cell to be used, and it is believed that for all proposed NK cells antibodies that bind cell surface molecules, preferably as described herein, can be identified.

To render a cancer-derived cell more acceptable for therapeutic use, it is generally pre-treated in some way to reduce or remove its propensity to form tumours in the patient. Specific NK cells used in examples are safe because they have been rendered incapable of division; they are irradiated and retain their killing ability but die within about 3-4 days. Treatments of potential NK cells for use in the methods herein include irradiation to prevent them from dividing and forming a tumour in vivo and genetic modification to reduce tumourigenicity, e.g. to insert a sequence encoding a suicide gene that is activatable to prevent the cells from dividing and forming a tumour in vivo. Suicide genes can be turned on by exogenous, e.g. circulating, agents that then cause cell death in those cells expressing the gene.

Specific embodiments of the invention use the NK cell line, KHYG-1. A derivative thereof can also be used, derived e.g. as per the modifications described immediately above or derived by culture. In a further specific embodiment, the NK cell line NK-92, or a derivative thereof, is used.

These cells can be used on their own in certain methods of the invention. Hence according to the invention a method of treating a tumour in a patient comprises administering an effective amount of KHYG-1 cells to the patient. The method of treating a tumour in a patient generally comprises administering an effective amount of a NK cell to the patient, wherein an antibody in the patient binds an antigen on the surface of the NK cell and the antibody further binds to an Fc receptor on a cell of the tumour. Preferred and optional aspects of earlier methods of the invention form preferred and optional aspects of these methods.

Still further, the invention provides the described antibodies, NK cells and combinations thereof for uses described herein. Thus the invention provides an antibody for use in treating a tumour in a patient, wherein the antibody binds an antigen on the surface of a natural killer (NK) cell and the antibody binds to an Fc receptor on a cell of the tumour. The invention provides an antibody and a NK cell for use in combination for treating a tumour in a patient, wherein the antibody binds an antigen on the surface of the NK cell and the antibody binds to an Fc receptor on a cell of the tumour. The invention provides KHYG-1 cells or derivatives thereof for use in treating a tumour in a patient. The invention also provides a NK cell for use in treating a tumour in a patient, wherein an antibody in the patient binds an antigen on the surface of the NK cell and the antibody binds to an Fc receptor on a cell of the tumour.

Still further, the invention provides a composition comprising (a) a NK cell, and (b) an antibody that binds to the NK cell.

The composition is for use as described elsewhere herein, and thus is suitable, for treatment of tumours, e.g. tumours of blood cells or solid tumours, suitable for treatment of cancers. In specific embodiments the composition is for treatment of leukemia, including acute myeloid leukemia.

Antibodies in the compositions are typically as elsewhere described, and hence may bind an Fc receptor selected from CD16, CD32 or CD64, and may bind a NK cell cytotoxicity receptor, e.g. NKp30 and NKp44. Other features of the antibodies are as described in relation to methods and uses of the invention, and are not repeated here.

Similarly, other optional and preferred features of the NK cells of the composition are as described elsewhere in relation to other methods and uses of the invention.

### EXAMPLES

The present invention is now described in more and specific details in relation to the use of the NK cell line KHYG-1 in causing leukemia cell death in humans, and to the enhancement of this effect through R-ADCC via NKp30 and NKp44 and presence on the target cell of CD32 that binds Fc regions on the antibodies. The invention is illustrated in the following examples, with reference to the accompanying drawings, in which:

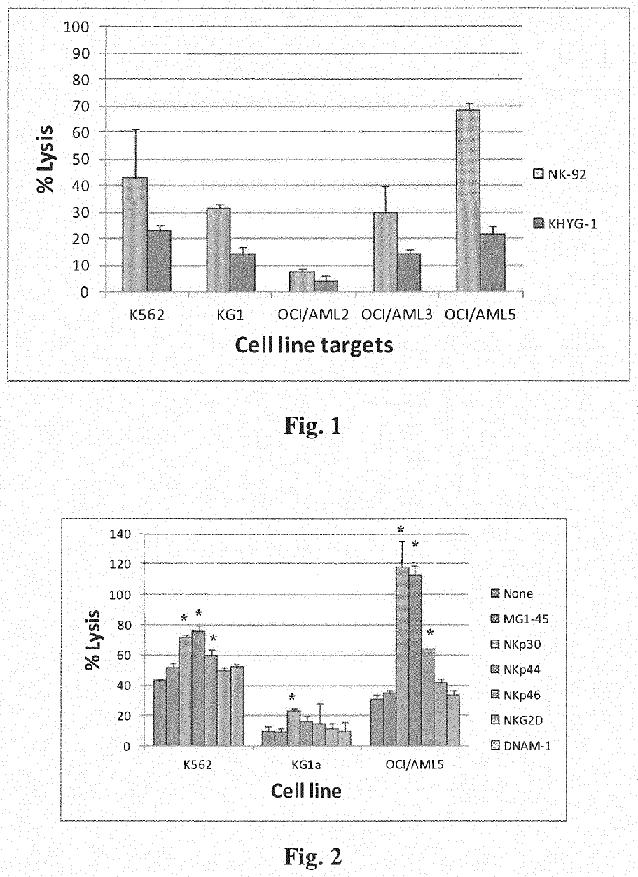

FIG. 1 shows NK-92 and KHYG-1 cytotoxicity against a panel of leukemia cell lines;

FIG. 2 shows the effect of antibody pre-treatment of activating receptors on KHYG-1 cytotoxicity against leukemia cell lines;

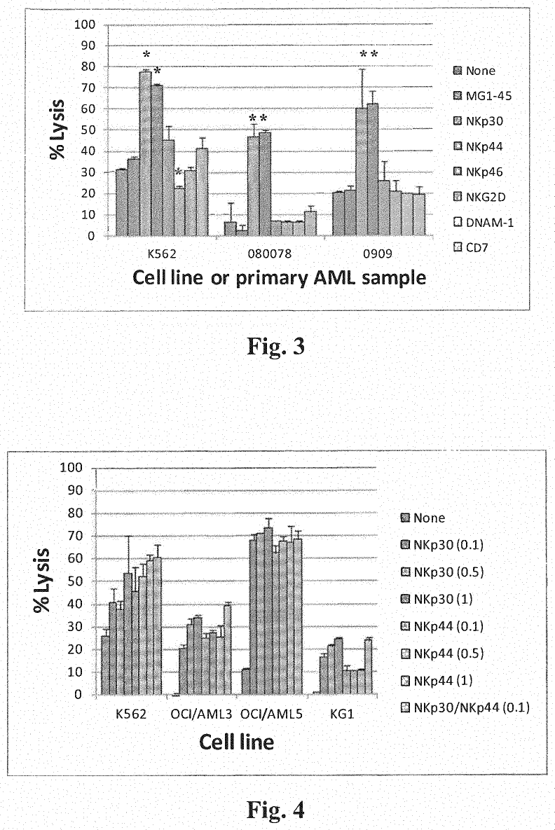

FIG. 3 shows the effect of antibody pre-treatment of activating receptors on KHYG-1 cytotoxicity against primary AML samples;

FIG. 4 shows the effect of antibody pre-treatment with varying concentrations of anti-NKp30 and anti-NKp44 on KHYG-1 cytotoxicity against leukemia cell lines;

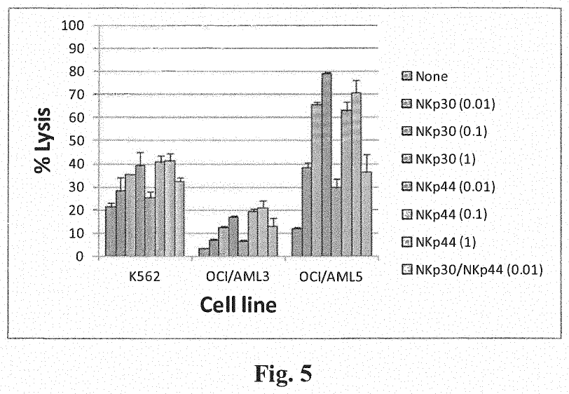

FIG. 5 shows the effect of antibody pre-treatment with varying concentrations of anti-NKp30 and anti-NKp44 on KHYG-1 cytotoxicity against leukemia cell lines;

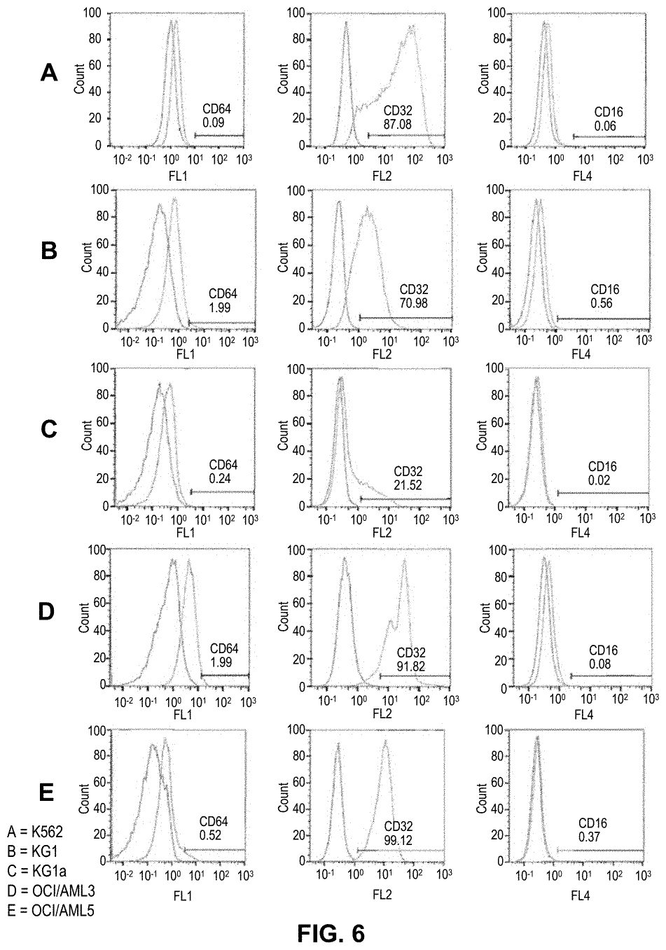

FIG. 6 shows Fc gamma receptor expression on leukemia cell lines (K562, KG1, KG1a, OCI/AML3, OCI/AML5);

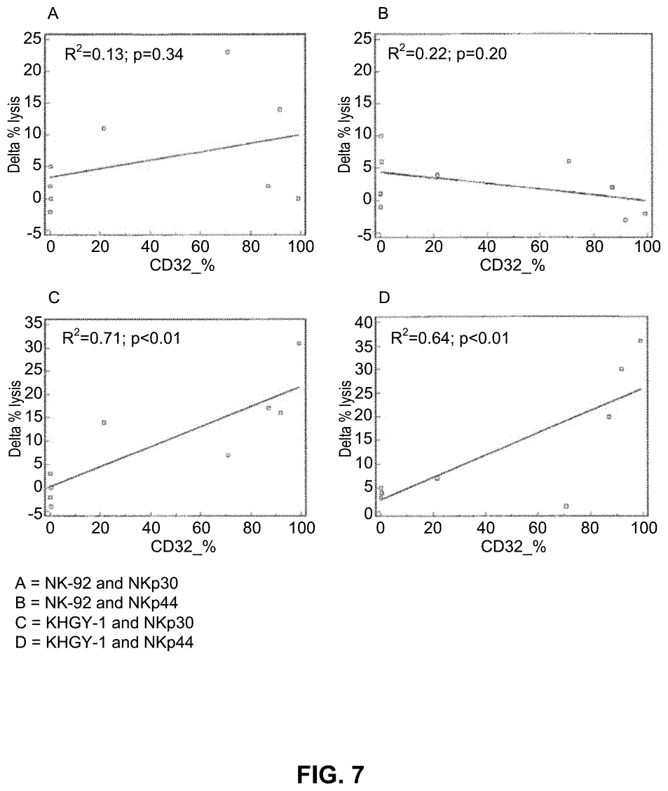

FIG. 7 shows regression analysis of CD32 expression and delta cytotoxicity of NKp30 and NKp44 pretreated NK-92 and KHYG-1 cells;

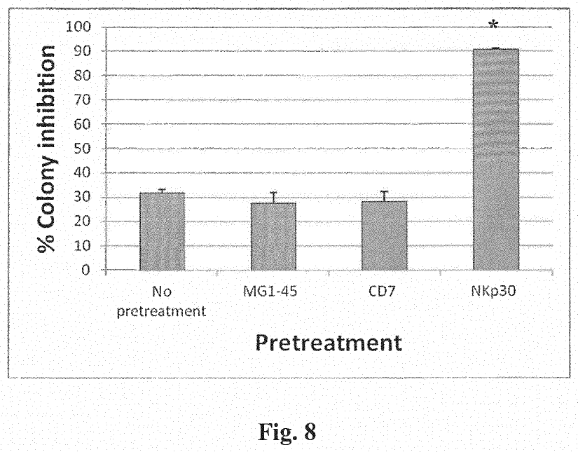

FIG. 8 shows a methylcellulose cytotoxicity assay of KHYG-1+/−pretreatment with antibodies against OCI/AML5;

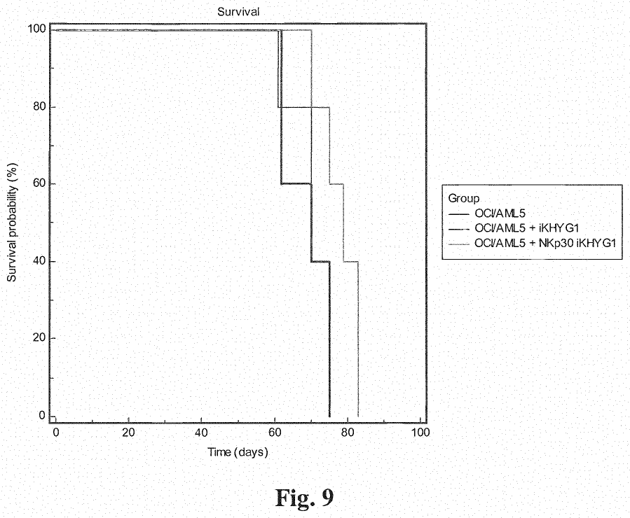

FIG. 9 shows the in vitro incubation of OCI/AML5 with iKHYG-1+/−anti-NKp30 and in vivo proliferation in NSG mice;

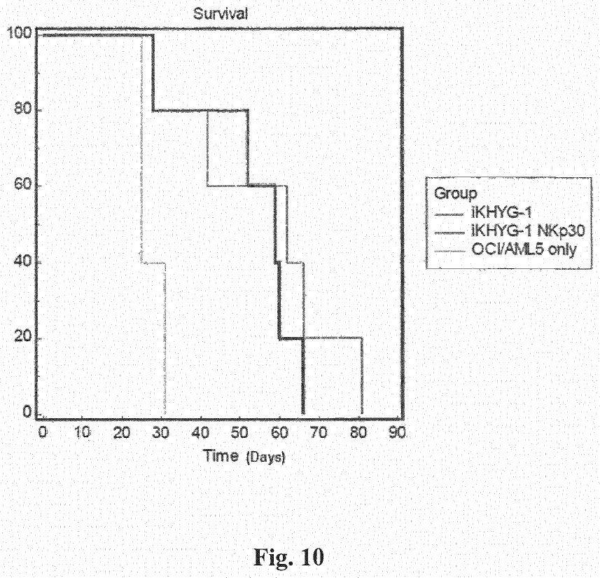

FIG. 10 shows the treatment of OCI/AML5 leukemia in NSG mice with iKHYG-1+/−NKp30 pretreatment;

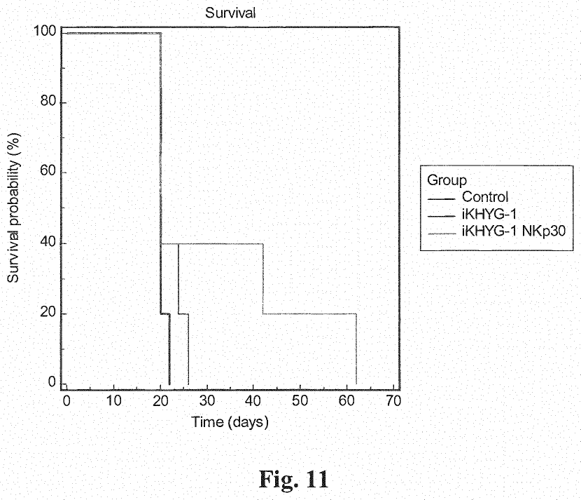

FIG. 11 shows the treatment of primary AML xenografted NSG mice with iKHYG-1+/−NKp30 pretreatment;

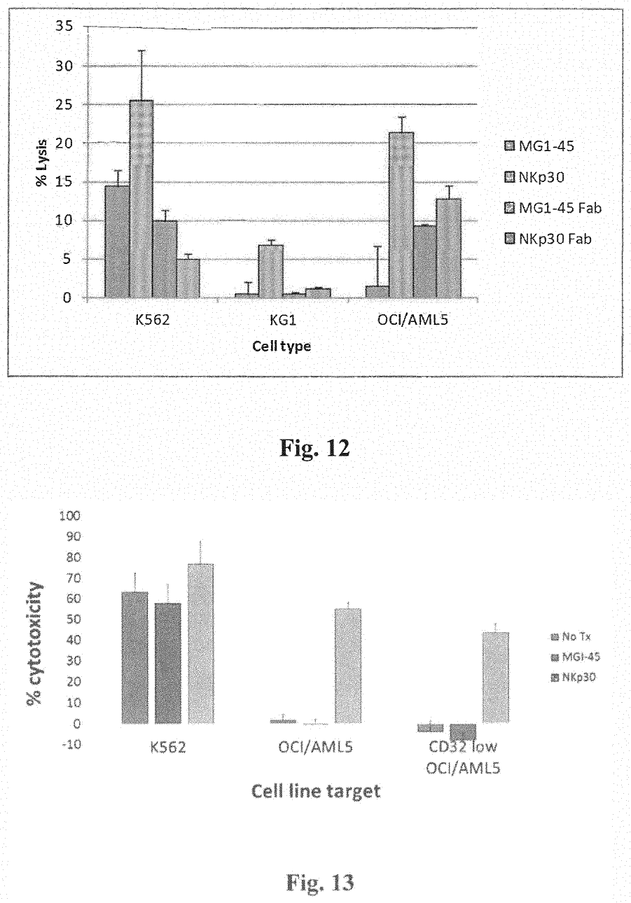

FIG. 12 compares the effect of pre-treatment of KHYG-1 with anti-NKp30 antibody and Fab fragments of the antibody on cytotoxicity against leukemia cell lines; and

FIG. 13 compares the cytotoxicity of anti-NKp30-coated KHYG-1 cells against unsorted OCI/AML5 cells and CD32^{low} OCI/AML5 cells.

In this example of the invention pretreatment of NK cell lines with monoclonal antibodies to activating receptors is demonstrated to cause several-fold enhancement of cytotoxicity against leukemic cell lines and primary AML blasts. This effect was most prominent with anti-NKp30 and anti-NKp44 pretreatment of KHYG-1 against CD32-expressing targets. Further specific work to eliminate other (otherwise credible) explanations showed R-ADCC as the mechanism of enhancement.

We further demonstrated an impact of NKp30 pretreated KHYG-1 in an in vivo model.

### Methods

### Cell Lines and Primary Samples

K562 was obtained from the ATCC and maintained in IMDM+20% FBS and 10% fetal bovine serum (FBS), respectively. KG1 and KG1a was obtained from the ATCC and maintained in IMDM+20% FBS and 10% FBS, respectively. OCI/AML 2, 3 and 5 were derived at the Ontario Cancer Institute (OCI). OCI/AML 2 and 3 were cultured in MEM alpha+10% FBS and OCI/AML5 was cultured in MEM alpha+10% FBS and 10% 5637 bladder carcinoma condition medium. KHGY-1 was purchased from The Human Science Research Resources Bank (JCRB0156; Tokyo, Japan) and cultured in GM1 (Ex Vivo medium with 450 U/ml and human A/B serum). NK-92 was obtained from Dr. Hans Klingemann and also cultured in Ex Vivo with human A/B serum and 450 U/ml of IL-2 (GM1). KHYG-1 was irradiated (iKHYG-1) with 1000 cGy prior to use in in vivo experiments. Five primary AML samples were obtained from the Princess Margaret Hospital Leukemia Tissue Bank as per institutional protocol (5890, 080179, 080078, 080008, 0909).

### Chromium Release Assay

We utilized a standard chromium release assay as previously described by our group (Williams, Wang et al. 2010) and detailed in the Chapter 2 methods section. Briefly, 1×10⁶ target cells were labelled with 100 μCi of Na₂⁵¹CrO₄ for 2 hours prior to plating 10 000 cells per wells followed by treatment with NK-92 at various concentrations. The amount of ⁵¹Cr present in supernatants was determined using a gamma counter and percent lysis calculated.

### Antibody Pretreatment of NK Cell Effectors

All antibodies used were from BIOLEGEND®. For NK pretreatment experiments, antibodies against the following NK receptors were utilized (clone; product #): NKp30 (clone P30-15; 325204), NKp44 (clone P44-8; 325104), NKp46 (clone 9E2; 331904), DNAM-1 (clone DX-11; 316802), NKG2D (clone 1D11; 320810), CD7 (CD7-6B7; 343102). Isotype controls specific to trinitrol phenol+KLH were utilized: MG1-45 (clone MG1-45; 401404) and MG2a-52 (clone MG2a-53, 401502). Briefly, 1.5×10⁶ NK cells (NK-92 or KHYG-1) were treated with 1 ml of AIM-V serum free medium for 1 hour, washed in 10 ml of AIM-V medium and resuspended in 1.5 ml of AIM-V medium (1×10⁶/m). Concentration of antibodies ranged from 10 μg/ml to 0.01 μg/ml. 0.1 μl (10⁵ cells) of NK cell suspension was added to 10 000 tumour targets also in AIM-V medium in 96 well U bottom plates to yield a 10:1 E:T ratio.

### Flow Cytometry

Immunophenotyping of BM was done using an FC500. FACS buffer was made with PBS+2 mM EGTA+2% FBS. For routine flow cytometry of leukemia and esophageal cancer cell lines the following antibodies to Fcγ receptors were utilized: CD16 APC (clone 3G8, 302011), CD32 PE (clone FUN-2, 303205), CD64 FITC (clone 10.1; 305005). Antibody concentrations were utilized at ˜1 μg/ml in a 50 μl reaction volume with 200 000 to 1,000,000 cells.

### High Throughput Sampling Flow Cytometry

Commercially validated FITC, PE or APC conjugated antibodies (374) to cell surface markers (BD PHARMINGEN™, EBIOSCIENCE™, ABCAM®, ABD SEROTEC®, BIOLEGEND®, LIFESPAN BIOSCIENCES™, MILTENYI™, R&D SYSTEMS™, BECKMAN COULTER®, and IMGENEX™) were aliquotted into individual wells of 96-well plates in Hanks Balanced Salt Solution supplemented with 1% bovine serum albumin and 2 mM EDTA (FACS buffer) at a dilution of 1:25 (Supplemental Table 1, 2). NK-92 or KHYG-1 cells (30×10e6) were prepared in 10 ml of PBS, spun down and resuspended in HBSS+1% BSA, 2 mM EDTA and volume adjusted to 1×106/ml. 50 μl of cell suspension (50 000 cells) suspension was added to each well to yield a final antibody dilution of 1:50. Cells were stained for 30 minutes on ice at a concentration of 0.25-1.0×106/mL, washed once with cold FACS buffer, and resuspended in FACS buffer with 0.1 μg/mL DAPI to allow for dead cell exclusion. Flow cytometry was performed using a High Throughput Sampler-equipped BECTON-DICKINSON™ LSRII flow cytometer. Plates were placed into an automated flow cytometry plate reader. Data was acquired and analyzed on FLOWJO 9™. Gating strategy utilized both a FS and SS plot and subsequent DAPI staining to exclude non-viable cells, followed by FSC-H and FSC-W to exclude doublets. Final gate was contoured around viable, unstained cells. Percentage positive cells and mean fluorescence intensity were quantitated for each marker.

### Animals

NOD/SCID gamma^{null} (NSG) mice from THE JACKSON LABORATORY™ were bred and maintained in the Ontario Cancer Institute animal facility according to protocols approved by the Animal Care Committee. Mice were fed irradiated food and BAYTRIL® (enrofloxacin) containing water ad libitum during experimental periods. Prior to infusion with AML NSG mice were irradiated with 200 cGy to facilitate engraftment. We developed ip and iv injection route OCI/AML5 NSG xenograft models utilizing a dose of 2×10⁶ cells. To determine impact of in vitro incubation with iKHYG-1 on proliferative capacity of OCI/AML5 the ip route of injection was utilized with sacrifice at humane endpoints. Briefly, OCI/AML5 cells were incubated in 15 ml conical tubes with or without iKHYG-1 (+/−1 μg/ml anti-NKp30 pretreatment×1 hour) at a 10:1 E:T ratio, spun at 1200 rpm to pellet and incubated for four hours at 37° C. Cell mixtures were then washed and resuspended in PBS and 2×10e6 OCI/AML5 cells with or without 20×10⁶ iKHYG-1 or NKp30 iKHYG-1 cells in 200 μl of PBS were injected ip into three cohorts of five NSG mice.

To determine in vivo the effect of NK cell line therapy OCI/AML5 or primary AML were injected iv on day 0 with and without iKHYG-1 or anti-NKp30 pretreated iKHYG-1 treatment started on day 3 (10×10⁶×6 doses; days 3, 5, 7, 10, 12, 14). The primary AML sample (080179) was derived from an M4 leukemia with aggressive engraftment features and passaged through NSG mice prior to use in these experiments.

### Calcein Cytotoxicity Assay

OCI/AML5 cells were knocked down for CD32 using three siRNA products targeting CD32 a, b and c. Subsequently, OCI/AML5 CD32 knocked down cells were sorted on a cell sorter to acquire the CD32 low fraction, setting acquisition gates to the bottom 10% population. NK cell cytotoxicity against OCI/AML5 and CD32 low OCI/AML5 cells lines was determined using the calcein cytotoxicity assay. Target cells were labeled with 10 μM of calcein-AM for 30 minutes, before 2 washes in serum free RPMI media. Cells were then resuspended at 5×104/ml in AIM-V serum free medium and 100 μl of the cell suspension added to a U bottom 96 well plate. Both NK-92 and CD16+IL-2+NK-92 cells were used as effector cells at a 10:1 Effector:Target (E:T) ratio. After effector cell addition to targets, plates were spun at 500 g for 1 min. Triton X (2%) was used to determine the maximum calcein release. Plates were incubated at 37° C. for 2 hours before 75 μl of the supernatant was transferred to a new plate for reading at 480/530 nm.

### Statistics

Survival analysis was done with Kaplan Meier survival curves using the log rank rest with MEDCALC™ software. Comparison of cytotoxicity and engraftment data was done using a two tailed student's t-test performed on MEDCALC™ software.

Linear regression analysis was done using MEDCALC™ software and used to generate scatter plots with best fit line, coefficients of determination (R²), F test and degree of significance.

### Results

### NK-92 and KHYG-1 Cytotoxicity Against Leukemia Cell Lines

NK-92 and KHYG-1 were tested against a panel of leukemic cell lines (K562, KG1, OCI/AML2, 3 and 5) at a 10:1 E:T ratio using the chromium release assay. Both cell lines demonstrated cytotoxicity against these targets, with NK-92 showing overall better cytotoxicity than KHYG-1 (FIG. 1). OCI/AML5 was particularly sensitive to NK-92 killing with percentage lysis of 68%, exceeding that for K562. OCI/AML2 was relatively resistant to killing by both cell lines with minimal cytotoxicity demonstrated.

NK-92 cytotoxicity against K562 was completely abrogated by calcium chelation at all effector targets ratios, indicating that granule exocytosis was the primary means of cytotoxicity. However, there was a small amount of residual killing of K562 by KHYG-1 particularly at effector:target ratios of 1:1 and 5:1.

Effect of Pretreating NK-92 and KHYG-1 with Activating Receptor Specific Antibodies

We attempted to address the role of common activating receptors in NK cell line-mediated recognition of leukemic targets by pretreating NK-92 and KHYG-1 with antibodies specific to NKp30, NKp44, NKp46, DNAM-1, NKG2D, and CD7 (10 μg/ml), prior to co-incubation with the target cells K562, KG1a and OCI/AML5. An off-target antibody was used as an isotype control. Pretreatment of NK-92 with antibodies to NKp30, NKp44 and NKp46 unexpectedly increased killing of K562 above isotype control [1.3× (p<0.0001), 1.2× (p<0.05), 1.2× (p=0.11)], while anti-NKp30 treatment enhanced killing of KG1a [+1.8× (p<0.0001)] and OCI/AML5 [1.2× (p<0.01)].

Treatment of KHYG-1 with antibodies to NKp30, NKp44 and NKp46 increased killing of K562 above isotype control [1.4× (p<0.001), 1.5× (p<0.01), 1.2× (p<0.05)], while anti-NKp30 treatment increased killing of KG1a [2.6× (p<0.01)] and anti-NKp30, anti-NKp44 and anti-NKp46 treatment increased killing of OCI/AML5 [3.4× (p<0.00001), 3.2× (p<0.0001), 1.8× (p<0.0001)] (FIG. 2). Pretreating NK cell lines with antibodies to DNAM-1 and NKG2D had minimal effects on cytotoxicity.

We then attempted a similar experiment with K562 and two primary AML cell lines. NK-92 and KHYG-1 were pre-treated with antibodies specific to NKp30, NKp44, NKp46, DNAM-1, NKG2D and CD7 (10 μg/ml) prior to co-incubation with the leukemic target cells K562, and the primary AML specimens 080078 and 0909. NK-92 cytotoxicity against primary AML samples demonstrated prominent increases above isotype control when pretreated with anti-NKp30 [7.1× (p<0.001) and 3.0× (p<0.0001)].

Pretreatment of KHYG-1 with either anti-NKp30 or anti-NKp44 led to dramatic increases of cytotoxicity relative to isotype control against primary AML samples 080078 [16.9× (p<0.0001) and 17.6× (p<0.001)] and 0909 [2.8× (p<0.0001) and 2.9× (p<0.001)] (FIG. 3). The dose dependence of anti-NKp30 and anti-NKp44 mediated enhancement of killing was then explored by testing several dose ranges.

In an attempt to determine the linear portion of the dose response curve and approximate the EC50%, pretreatment of NK cell lines was done with a range of doses of anti-NKp30 and anti-NKp44 (1, 5 and 10 μg/ml) against K562, OCI/AML3 and OCI/AML5 and primary AML 080078. A dose response was seen with anti-NKp30 pretreatment of NK-92 against OCI/AML3 and primary AML sample 080078 however the EC50% was less than the lowest dose used (1 μg/ml) (data not shown). KHYG-1 had a similar degree of enhancement seen at all dose ranges of both anti-NKp30 and anti-NKp44 antibody pretreatments (data not shown).

A lower dose range was then selected utilizing NK cell lines pretreated with isotype control, anti-NKp30 and anti-NKp44 at 0.1, 0.5 and 1 μg/ml. Isotype-control pretreated NK-92 and KHYG-1 had minimal effects on cytotoxicity against K562, OCI/AML3, OCI/AML5 and KG1, with no dose response. Anti-NKp30 pretreatment enhanced NK-92 cytotoxicity against OCI/AML3 (EC50%˜0.1 μg/ml) and KG1 (EC50%<0.1 μg/ml) only. Combined anti-NKp30 and anti-NKp44 pretreatment (0.1 μg/ml) of NK-92 did not have additive or synergistic effects on cytotoxicity against any targets.

Anti-NKp30 pretreatment enhanced KHYG-1 cytotoxicity against all targets with a dose response seen for K562 (EC50%˜0.1 μg/ml), OCI/AML3 (EC50%<0.1 μg/ml), and KG1 (EC50%<0.1 μg/ml), while OCI/AML5 showed a plateau from the lowest dose (EC50%<0.1 μg/ml). Anti-NKp44 pretreatment enhanced KHYG-1 cytotoxicity against all targets, with a dose response seen for K562 only (EC50%˜0.1 μg/ml), while OCI/AML3, KG1 and OCI/AML5 showed a plateau from the lowest dose (EC50%<0.1 μg/ml) (FIG. 4).

Combined anti-NKp30 and anti-NKp44 pretreatment (0.1 μg/ml) of KHYG-1 demonstrated greater enhancement on cytotoxicity than each antibody alone against K562, OCI/AML3 and KG1, but not OCI/AML5.

Not only did the combined dosing of 0.1 μg/ml anti-NKp30 and anti-NKp40 exceed the effect of each antibody alone at this dose level, it also matched or exceeded the effect of a 10-fold higher dose (1 μg/ml) of each antibody alone for K562, OCI/AML3 and KG1, demonstrating true synergy.

The % lysis values for untreated, isotype control, anti-NKp30, anti-NKp40 and combined anti-NKp30 and anti-NKp44 groups (0.1 μg/ml) and anti-NKp30 and anti-NKp44 (1 μg/ml) for the cell line targets treated with KHYG-1 are tabulated for comparison (Table 1).

TABLE 1
Comparison of antibody pretreatment effects on KHYG-1
cytotoxicity and synergy assessment at 0.1 μg/ml

| | % Lysis (mean +/− SD) | | | | | | |
|---|---|---|---|---|---|---|---|
| | Pretreatment | | | | | | |
| | 0.1 μg/ml | | | | | | |
| | | | | | Anti- | 1.0 μg/ml | |
| | | MG2a- | Anti- | Anti- | NKp44/ | Anti- | Anti- |
| Cell line | None | 53 | NKp30 | NKp44 | NKp30 | NKp30 | NKp44 |
| K562 | 26 +/− | 37 +/− | 41 +/− | 46 +/− | *61 +/− | 54 +/− | 59 +/− |
| | 3.2 | 2.9 | 6.0 | 11.1 | 5.9 | 16.7 | 2.6 |
| OCI/AML3 | 0 +/− | 9 +/− | 20 +/− | 25 +/− | *39 +/− | 34 +/− | 26 +/− |
| | 0.8 | 7.8 | 2.0 | 1.5 | 1.7 | 1.3 | 4.8 |
| OCI/AML5 | 11 +/− | 19 +/− | 68 +/− | 62 +/− | 69 +/− | 74 +/− | 67 +/− |
| | 0.5 | 3.1 | 2.7 | 3.5 | 3.8 | 42 | 7.0 |
| KG1 | 1 +/− | 9 +/− | 16 +/− | 10 +/− | *24 +/− | 25 +/− | 11 +/− |
| | 0.4 | 1.7 | 1.7 | 2.2 | 1.1 | 0.5 | 0.7 |

*Combined anti-NKp30 and anti-NKp44 regimens that yielded statistically significant (p < 0.05) increases above antibodies alone in separate comparisons are in bold font.

While dose responses could be seen in the range of 0.1 to 1 μg/ml in many cases, the lowest dose exceeded the EC50%. Therefore, an additional experiment testing a dose range one log lower was conducted (0.01, 0.1 and 1 μg/ml). There was minimal effect of the isotype control (MG2a-53) antibody in this range, with no dose response seen (data not shown). Pretreatment of NK-92 with 0.01, 0.1 and 1 μg/ml of anti-NKp30 enhanced cytotoxicity of OCI/AML3 only (ED50% 0.01 to 0.1 μg/ml) and there was no effect of anti-NKp44 pretreatment.

Anti-NKp30 and anti-NKp44 pretreatment enhanced KHYG-1 cytotoxicity against all targets with a dose response seen for K562, OCI/AML3 and OCI/AML5 (EC50% 0.01-0.1 μg/ml).

Combined anti-NKp30 and anti-NKp44 pretreatment of KHYG-1 (0.01 μg/ml) demonstrated synergistic effects on cytotoxicity against OCI/AML3 only at this dose level.

The combined dosing of 0.01 μg/ml anti-NKp30 and anti-NKp40 exceeded the effect of each antibody alone (2×) and the absolute cytotoxicity was comparable to a 10-fold higher dose of each antibody (0.1 μg/ml). The % lysis values for untreated, isotype control, anti-NKp30, anti-NKp40 and combined anti-NKp30 and anti-NKp44 groups (0.01 μg/ml) and anti-NKp30 and anti-NKp44 (1 μg/ml) for the cell line targets treated with KHYG-1 and its isotype controls are tabulated for comparison (Table 2).

TABLE 2
Comparison of antibody pretreatment effects on KHYG-1
cytotoxicity and synergy assessment at 0.01 μg/ml

| | % Lysis (mean +/− SD) | | | | | | |
|---|---|---|---|---|---|---|---|
| | Pretreatment | | | | | | |
| | 0.01 μg/ml | | | | | | |
| | | | | | Anti- | 0.1 μg/ml | |
| | | MG2a- | Anti- | Anti- | NKp44/ | Anti- | Anti- |
| Cell line | None | 53 | NKp30 | NKp44 | NKp30 | NKp30 | NKp44 |
| K562 | 21 +/− | 29 +/− | 28 +/− | 26 +/− | 32 +/− | 35 +/− | 41 +/− |
| | 1.6 | 2.4 | 6.0 | 2.5 | 2.0 | 0.5 | 3.4 |
| OCI/AML3 | 4 +/− | 7 +/− | 7 +/− | 7 +/− | 13* +/− | 13 +/− | 19 +/− |
| | 0.1 | 1.8 | 0.8 | 0.6 | 3.8 | 0.5 | 3.4 |
| OCI/AML5 | 12 +/− | 16 +/− | 38 +/− | 30 +/− | 36 +/− | 65 +/− | 63 +/− |
| | 0.8 | 5.0 | 1.9 | 3.8 | 7.6 | 1.5 | 5.4 |
| KG1 | 9 +/− | 9 +/− | 6 +/− | 9 +/− | 11 +/− | 9 +/− | 15 +/− |
| | 7.4 | 1.2 | 1.0 | 4.7 | 1.0 | 1.1 | 4.5 |

Combined anti-NKp30 and anti-NKp44 regimens that yielded statistically significant (p < 0.05) increases above antibodies alone in separate comparisons are in bold font.

To determine if anti-NKp30 and anti-NKp44 pretreatment of NK cell lines could enhance cytotoxicity against a solid tumour, we performed the same experiment with esophageal cancer cell lines (FLO-1, OE-33, SKGT-4, KYAE-1). However, pretreatment of NK-92 and KHYG-1 with 0.1 μg/ml of anti-NKp30 and anti-NKp44 mAb did not enhance cytotoxicity against four esophageal cancer cells lines relative to the isotype control. This suggested the presence of a unique cell surface marker present on leukemia cells, but not esophageal cancer cells, responsible for mediating the enhancing effect of antibody-pretreated NK cell lines.

### Relationship of Fcγ Receptor Expression and Enhancement of Cytotoxicity

We conducted HTS flow cytometry on two representative leukemic cell line targets (OCI/AML3 and OCI/AML5) to assess for potential cell surface markers that might be involved in enhancing the cytotoxicity of anti-NKp30 or anti-NKp44 coated NK cell lines. We noted a high degree of CD32 (FcγRII) expression on both cell lines. Subsequently, we conducted routine flow cytometry on all leukemic and esophageal cancer cell lines for expression of Fcγ receptors (CD16, CD32 and CD64). The leukemia cell lines showed relatively high expression of Fcγ receptor II (CD32), but very low expression of Fcγ receptor I (CD64) or Fcγ receptor III (CD16) on leukemia lines K562, KG1, KG1a, OCI/AML3, and OCI/AML5.

The histogram shape for K562 suggested the presence of both intermediate and high CD32 expressing subpopulations. KG1a appeared to have a dual population of negative and low CD32 expressing subpopulations. OCI/AML3 had a clear dual peak representing two high CD32 expressing subpopulations. There were clear single populations of CD32 expressing cells in KG1 (moderate) and OCI/AML5 (high). There was no significant expression of Fcγ receptors on esophageal cancer lines (OE-33, FLO-1, KYAE-1 and SKGT-4).

The percent positivity of each leukemic and esophageal cancer cell line was determined from flow cytometry plots. Data from prior cytotoxicity assays measuring the enhancement of cytotoxicity of NK-92 and KHYG-1 when pretreated with 10 μg/ml of either anti-NKp30 or anti-NKp44 antibody were compared to isotype controls and delta cytotoxicity calculated. The delta cytotoxicity relative to isotype control was correlated with the degree of CD32 expression using regression analysis to create best fit lines, calculate co-efficient of determination (R²) and statistical significance. Regression analysis of the relationship of delta cytotoxicity of antibody-pretreated NK-92 with CD32 expression of targets did not reveal a correlation for anti-NKp30 (R²=0.13; p=0.34) and anti-NKp44 (R²=0.22; p=0.20) pretreatments (A and B).

However, regression analysis of the relationship of delta cytotoxicity of antibody pretreated KHYG-1 with CD32 expression of targets revealed a correlation for both anti-NKp30 (R²=0.71; p<0.01) and anti-NKp44 pretreatments (R²=0.64; p<0.01) (C and D).

### Effect of Anti-NKp30 Pretreatment on NK Cell Line Cytotoxicity Against Clonogenic OCI/AML5

To determine the effect of anti-NKp30 and anti-NKp44 pretreated NK cell lines against clonogenic leukemic cells, we utilized our previously established clonogenic cytotoxicity assay utilizing OCI/AML5 as the target. Comparison of killing was made with untreated, isotype control (MG1-45) and anti-CD7 pretreated NK cell lines. CD7 is highly expressed on NK-92 and KHYG-1, with no confirmed activating capacity in these cell lines. Therefore, anti-CD7 antibody pretreatment was chosen as an additional control. There was no difference between isotype control and CD7-pretreated NK-92. Pretreatment of NK-92 with 0.1 μg/ml anti-NKp30 had only a slight impact (+3.7%; p<0.05) on OCI/AML5 clonogenic capacity relative to baseline and isotype control.

However, pretreatment of KHYG-1 with anti-NKp30 enhanced % colony inhibition 3-fold (+63.1%; p<0.0001) relative to baseline and isotype control (FIG. 8). While the baseline % colony inhibition of NK-92 (61.9%) was greater than KHYG-1 (32.0%), anti-NKp30 pretreated KHYG-1 (90.7%) had the greater inhibition relative to NK-92 (+28.8%; p<0.0001).

In Vitro Effect of Anti-NKp30 Pretreated iKHYG-1 Against OCI/AML5 Capacity for Leukemic Progression in an NSG Xenograft Model

We tested the in vitro cytotoxic effect of KHYG-1 on in vivo progression of leukemia in an OCI/AML5 xenograft model. KHYG-1 proliferation was prevented by irradiation with 1000 cGy prior to use. OCI/AML5 cells were co-incubated with irradiated KHYG-1 (iKHYG-1)+/−1 μg/ml anti-NKp30 pretreatment for one hour prior to a 4 hour co-incubation at a 10:1 E:T with OCI/AML5 cells. Subsequently, cell mixtures were injected ip into three cohorts of NSG mice with survival as an endpoint. Individual NSG mice were injected with 2×10⁶ OCI/AML5 cells+/−20×10⁶ viable effector cells (iKHYG-1 or anti-NKp30 pretreated iKHYG-1). At 9 weeks, control mice developed progressive malignant ascites with minimal splenomegaly and imbedded vascular tumours in the momentum.

Co-incubation with iKHYG-1 did not improve survival (p=0.92). However, anti-NKp30 pretreated iKHYG-1 improved survival compared to the no therapy (p<0.05) or iKHYG-1 (p<0.05) cohorts.

Effect of Anti-NKp30 Pretreatment of iKHYG-1 on Therapeutic Efficacy for OCI/AML5 or Primary AML Xenografted Mice

We evaluated OCI/AML5 engraftment potential in NSG mice by infusing 5×10⁶ OCI/AML5 cells via tail vein and measured bone marrow engraftment at two weeks. Bone marrow engraftment of OCI/AML5 was detected by measuring human CD33 expression in bone marrow samples and revealed relatively rapid, but variable bone marrow engraftment (13.0, 52.3, 29.9, 63.5%). In a subsequent survival endpoint experiment, 2×10⁶ OCI/AML5 cells were injected iv into cohorts of five mice. OCI/AML5-xenografted mice were then treated without and with iKHYG-1 or anti-NKp30 pretreated iKHYG-1 (10×10⁶×6 doses ip). There was significant improvement in survival of mice treated with either iKHYG-1 (+35 days median survival; p<0.05) or anti-NKp30 pretreated iKHYG-1 (+37 day median survival p<0.05) above control.

We utilized a primary AML sample (080179) known to engraft and cause leukemia in NSG mice as a model to test the efficacy of irradiated NK-92 and KHYG-1 pretreated with and without anti-NKp30 (1 μg/ml) prior to injection into NSG mice inoculated with primary AML. iKHYG-1 and iNK-92 did not prolong survival in the primary AML model, although iKHYG-1 pretreated with anti-NKp30 showed some longer term survivors (3-4 weeks above control median) with a trend toward significance (p=0.20) versus iKHYG-1 alone (FIG. 11).

### Elucidation of Cytotoxicity Mechanism

Work thus far demonstrated that pretreatment of KHYG-1 with anti-NKp30 antibodies lead to greatly enhanced cytotoxicity against several cell lines and primary AML targets. However, there are many potential mechanisms that could explain this experimental data, leading to doubt as to the utility of the observations and as to application thereof in therapy.

We devised further experimental work to divine the actual mechanism. Specifically, Fab fragments of the anti-NKp30 (P30-15) clone and isotype control (MG1-45) were generated. KHYG-1 was precoated with 0.1 μg/ml of anti-NKp30 and isotype control whole antibody or Fab fragments for 1 hour and then washed in AIM-V medium. Precoated KHYG-1 was then used in a four hour chromium release assay against K562, KG1 and OCI/AML5 targets (FIG. 12). Only anti-NKp30 antibody was able to significantly enhance cytotoxicity against the three cell lines, while the anti-NKp30 antibody fragment either blocked cytotoxicity (K562), had no impact on cytotoxicity (KG1) or had minimal effects above isotype control Fab (OCI/AML5).

We further devised an experiment in which Fc receptor expression on the target cells was reduced. Specifically, to determine target CD32 dependence of the antibody-mediated enhancement of KHYG-1 cytotoxicity, as proof R-ADCC is the mechanism responsible, we attempted to use siRNA knockdown of the three gene isoforms of CD32 (a, b and c). While we were unable to achieve complete CD32 knockdown in the OCI/AML5 cell line, we were able to use cell sorting to acquire a CD32 low OCI/AML5 population for subsequent testing in a calcein cytotoxicity assay.

Unsorted OCI/AML5 targets were resistant to KHYG-1 cells, unless they were pretreated with 0.1 μg/ml of anti-NKp30 antibody, leading to a high degree of cytotoxicity at a 10:1 effector:target ratio, near equivalent to the NK sensitive K562 cell line. While CD32 low OCI/AML5 were sensitive to anti-NKp30 pretreated KHYG-1 cells, the degree of enhancement was 12% less (p=0.02) than for unsorted OCI/AML5 (FIG. 13).

In the context of this culture environment, in which the NK cells are localized with the target cancer cells, this is a highly significant effect and demonstrates that the enhancement in cytotoxicity is dependent on Fc gamma receptor II (CD32) expression on the target, further supporting R-ADCC as the mechanism of antibody enhanced cytotoxicity of KHYG-1 cells.

Therefore, the mechanism of enhancement was determined to be R-ADCC and not co-activation, co-stimulation or any other effect. Progress in developing therapies based on the totality of the work herein was thus made possible.

### Discussion

Here, we have conducted all experiments with KHYG-1 using a clinical grade medium (GM1) that lacks fetal bovine serum and may be suitable for future clinical application. KHYG-1 cytotoxicity against primary AML has not been previously reported. We noted that KHYG-1, without antibody, was less effective than NK-92 at killing both leukemia cell lines and primary AML samples.

We sought to determine the effect of pretreating NK-92 and KHYG-1 with antibodies against a panel of activating receptors commonly associated with NK cell cytotoxicity (natural cytotoxicity receptors, NKG2D and DNAM-1). While we anticipated potential blocking of cytotoxicity at 10 μg/ml against a panel of leukemia cell line targets, we observed stimulation of cytotoxicity from some of the antibodies and no blocking of cytotoxicity. Treatment of NK-92 with antibodies to NKp30, NKp44 and NKp46 increased killing of K562 by approximately 10%, while only anti-NKp30 treatment enhanced killing of KG1a and OCI/AML5. Further, anti-NKp30, but not anti-NKp44 pretreated NK-92 had a large degree of enhancement in killing of primary AML.

HTS flow cytometry demonstrated higher expression of NKp30, NKp44 and NKp46 on KHYG-1 compared with NK-92. Treatment of KHYG-1 with antibodies to all the natural cytotoxicity receptors increased killing of K562 and OCI/AML5 to a greater degree than NK-92. KHYG-1 cytotoxicity against primary AML was enhanced to a greater degree than NK-92 with pretreatment with anti-NKp30 or anti-NKp44. Pretreatment of NK cell lines with antibodies against DNAM-1, NKG2D (both commonly involved in NK cell recognition) induced small statistically significant inhibitory effects against target K562 in some experiments, indicating a possible role for these molecules in recognition. However, anti-DNAM-1 and anti-NKG2D were not able to facilitate reverse ADCC despite high expression of DNAM-1 and NKG2D on both cell lines.

We sought to determine the dose response curve of antibody-mediated cytotoxic enhancement, which was most prominent with KHYG-1 against OCI/AML5. Increases in KHYG-1 cytotoxicity against OCI/AML5 was seen as low as 0.01 μg/ml, which is near the EC50 of the stimulatory effect. A plateau in enhancement started to occur at 0.1 μg/ml with slight increase in efficacy at 1 μg/ml. NK-92 had lesser enhancement than KHYG-1 with this approach, but effects could be observed in the 0.01 μg/ml dose range particularly against OCI/AML3, which is somewhat resistant to NK-92 cytotoxicity.

The antibodies we used were reported as blocking antibodies, based on studies of endogenous NK cell cytotoxicity (Markel, Seidman et al. 2009)—inconsistent with the results observed. Fab fragments of the anti-NKp30 (P30-15) clone and isotype control (MG1-45) were generated. KHYG-1 was precoated with anti-NKp30 and isotype control whole antibody or Fab fragments of each, before co-incubating with K562, KG1 and OCI/AML5 targets. Only anti-NKp30 antibody was able to significantly enhance cytotoxicity against the three cell lines.

Moreover, a population of OCI/AML5 target cells were selected for low expression of CD32, before being co-cultured with KHYG-1 cells, pre-coated with anti-NKp30 antibody. The ability of the anti-NKp30-coated KHYG-1 cells to kill the CD32^{low}OCI/AML5 target cells was significantly reduced, when compared to a group of unsorted OCI/AML5 cells expressing higher levels of CD32.

These unexpected but advantageous findings show that the increased cytotoxicity was via reverse ADCC.

The R-ADCC mechanism responsible for the increased cytotoxicity observed was further confirmed via the finding that four esophageal cell lines, known to express no or low levels of Fc receptors, were not responsive to treatment with KHYG-1 cells, pre-coated with either anti-NKp30 antibody or anti-NKp44 antibody.

Hence, the invention uses R-ADCC as a means of enhancing NK cell line cytotoxicity—in examples against leukemic cell lines and, more importantly, primary AML cells, but also of wider application.

We then sought to determine if the enhancing effect of pretreating NK cell lines with anti-NKp30 and anti-NKp44 held against clonogenic cells using our established methylcellulose cytotoxicity assay. We noted that NK-92 was relatively effective at inhibiting OCI/AML5 colony formation, but this could not be enhanced by pretreatment with anti-NKp30 over isotype control. However, KHYG-1 was less effective at inhibiting OCI/AML5 colony formation alone, but this could be enhanced three-fold by pretreatment with anti-NKp30 antibody. The inhibition of colonies indicates a cytotoxic or cytostatic effect on clonogenic cells within the cell line populations that represent leukemic stem and progenitor cells. Therefore, this provided evidence that reverse ADCC facilitated cytotoxicity against leukemic stem cells.

To evaluate the impact of NK cell line therapy in vivo, we established an OCI/AML5 xenograft model in NSG mice. OCI/AML5 was derived from a patient with M4 leukemia, and highly expresses CD33, which is useful for tracking in vivo (Wang, Koistinen et al. 1991). We confirmed injection of OCI/AML5 iv led to leukemia with splenomegaly and bone marrow engraftment. However, ip injection tended to cause progressive malignant ascites over a longer timeframe, rather than classic leukemia.

To determine the effect of in vitro cytotoxicity on in vivo proliferation, we incubated OCI/AML5 with or without iKHYG-1 (+/−anti-NKp30 pretreatment) and injected the cells ip. We utilized this injection route instead of iv, because of the high cell load (2×10⁶ OCI/AML5+20×10⁶ viable iKHYG-1+˜3×10⁶ non-viable iKHYG-1) and relatively larger KHYG-1 cells, which might have caused pulmonary stress to the mice if injected via tail vein. Co-incubation with iKHYG-1 had no impact, while anti-NKp30 pretreated iKHYG-1 improved median survival by 10 days.

We subsequently utilized the iv injection OCI/AML5 NSG xenograft model to test therapeutic efficacy of iKHYG-1 with or without anti-NKp30 pretreatment. Unexpectedly, iKHYG-1 was able to improve the median survival by 35 days, despite its poor cytotoxicity in the CRA against bulk OCI/AML5. However, KHYG-1 had three-fold better cytotoxicity against clonogenic OCI/AML5 than bulk OCI/AML5, as determined by the CRA, providing some basis for this finding. This is the first demonstration of efficacy by KHYG-1 in an in vivo cancer model and confirms that the irradiated cells can persist and reduce tumour burden. This is also the first evidence that the irradiated cells can function in vivo. We then used a primary AML xenograft model to test iKHYG-1 and anti-NKp30 iKHYG-1, demonstrating lack of efficacy of iKHYG-1, but a trend to improved survival in the anti-NKp30 iKHYG-1 treated group.

In summary, we demonstrated that NK-92 and KHYG-1 have cytotoxicity against a broad range of leukemic targets that can be enhanced several-fold by anti-NKp30 and anti-NKp44 antibodies. For KHYG-1, cancer cell killing was achieved via R-ADCC via interaction of antibody coated effectors with FcγRII (CD32) on the target cells. Furthermore, antibodies to NK cell surface markers enhanced cytotoxicity of KHYG-1 against clonogenic leukemic cells (cancer stem cells) and reduced in vivo proliferation of leukemia.

The invention hence provides methods, uses and compositions for treatment for tumours using antibodies, NK cells or both in combination via R-ADCC.

### ADDITIONAL REFERENCES

Notter, M., W. D. Ludwig, et al. (1993). “Selective targeting of human lymphokine-activated killer cells by CD3 monoclonal antibody against the interferon-inducible high-affinity Fc gamma RI receptor (CD64) on autologous acute myeloid leukemic blast cells.” Blood 82(10): 3113-3124.
Pende, D., S. Parolini, et al. (1999). “Identification and molecular characterization of NKp30, a novel triggering receptor involved in natural cytotoxicity mediated by human natural killer cells.” Journal of Experimental Medicine 190(10): 1505-1516.
Saxena, R. K., Q. B. Saxena, et al. (1982). “Identity of effector cells participating in the reverse antibody-dependent cell-mediated cytotoxicity.” Immunology 46(2):459-464.
Lee, H. W. et al. (2014). “Evaluation of therapeutic effects of natural killer (NK) cell-based immunotherapy in mice using in vivo apoptosis bioimaging with a caspace-3 sensor.” The FASEB Journal 28(7):2932-2941.
Jing, Y. et al. (2015). “Identification of an ADAM17 cleavage region in human CD16 (Fc[gamma]RIII) and the engineering of a non-cleavable version of the receptor in NK cells.” PLOS One 10(3):e0121788.
Ferlazzo, G. et al. (2002). “Human dendritic cells activate resting natural killer (NK) cells and are recognized via the NKp30 receptor by anctivated NK cells.” 195(3):343-351.
Williams, B. A. (2010). “Clonogenic assays measure leukemia stem cell killing not dectectable by chromium release and flow cytometric cytotoxicity assays.” Cytotherapy 12(7):951-960.
Arnon, T. I. et al. (2008). “Harnessing soluble NK cell killer receptors for the generation of novel cancer immune therapy.” PLOS One 3(5):e2150.
Williams, B. (2010). “NK-92 preferentially targets acute myeloid leukemia stem cells.” Blood 116(21):1745.
Suck, G. et al. (2006). “Irradiated KHYG-1 retains cytotoxicity: potential for adoptive immunotherapy with a natural killer cell line.” International Journal of Radiation Biology 82(5):355-361.
Swift, B. et al. (2012). “Natural killer cell lines preferentially kill clonogenic multiple myeloma cells and decrease myeloma engraftment in a bioluminescent xenograft mouse model.” Haematologica—The Hematology Journal 97(7):1020-1028.
Kobayashi, E. et al. (2014). “Antibody-dependent cellular cytotoxicity and cytokine/chemokine secretion by KHYG-1 cells stably expressing Fc[gamma]RIIIA.” Immunology Letters 161(1):59-64.
Grier, J. T. et al. (2012). “Human immunodeficiency-causing mutation defines CD16 in spontaneous NK cell cytotoxicity.” Journal of Clinical Investigation 122(10):3769-3780.
Deng, X. (2014). “Ex vivo-expanded natural killer cells kill cancer cells more effectively than ex vivo-expanded [gamma] [delta] T cells.” International Immunopharmacology 22(2):486-491.
Muller, T. et al. (2014). “Allogeneic and xenogeneic anti-tumor effect of callithrix jacchus natural killer cells is dependent on KNp30 and B7-H6 interaction. Journal of Biological Regulators and Homeostatic Agents 28(2):183-193.
Swift, B. et al. (2011). “Efficacy of NK-92 against human multiple myeloma in a NOD/SCID gamma null mouse model.” Blood 118(21):807.

1. A method of treating a cancer in a patient via reverse antibody dependent cell-mediated cytotoxicity (R-ADCC), comprising administering an effective amount of an antibody to the patient, wherein
the antibody binds an antigen on the surface of a natural killer (NK) cell; and
the antibody binds to an Fc receptor on a cell of the cancer.

2. A method according to claim 1, wherein the antibody binds an Fc receptor selected from the group consisting of CD16, CD32, and CD64.

3. A method according to claim 1, wherein killing of cancer cells is achieved by R-ADCC.

4. A method according to claim 1, for treatment of blood cancer.

5. A method according to claim 1, for treatment of cancer by killing cancer stem cells.

6. A method according to claim 1, for treatment of leukemia.

7. A method according to claim 1, for treatment of B-cell lymphoma.

8. A method according to claim 1, for treatment of solid cancers.

9. (canceled)

10. A method according to claim 1, wherein the antibody binds to a NK cell surface marker selected from NKp30 and NKp44.

11. A method according to claim 1, for treating a tumour in a patient, comprising administering to the patient an effective amount of the antibody and an effective amount of NK cells, wherein
the antibody binds an antigen on the surface of the NK cells; and
the antibody binds to an Fc receptor on a cell of the tumour.

12. A method according to claim 11, comprising administering the NK cells prior to, simultaneously with or subsequent to administering the antibody.

13. A method according to claim 11, comprising administering the antibody bound to the NK cell.

14. A method according to claim 13, comprising pre-incubating the antibody with the NK cell prior to administering the antibody bound to the NK cell.

15. A method according to claim 11, wherein the NK cell is a NK cell obtained from a cancer cell line.

16. A method according to claim 15, wherein the cell derived from a cancer cell line is irradiated to prevent it from dividing and forming a tumour in vivo.

17. A method according to claim 15, wherein the NK cell comprises a suicide gene that is activatable to prevent it from dividing and forming a tumour in vivo.

18. A method according to claim 11, wherein the NK cell is a KHYG-1 cell or derivative thereof.

19.-53. (canceled)

54. A method of treating a blood cancer in a human patient, comprising administering an antibody to the patient, wherein
the antibody binds an antigen on a surface of a natural killer (NK) cell, said antigen being selected from the group consisting of NKp30, NKp44, NKp46, SLAMF-7, and NKG2D; and
an Fc portion of the antibody binds an Fc receptor on a cell of the cancer, said receptor being selected from the group consisting of CD16, CD32, and CD64; and
wherein killing of the cancer is via reverse antibody dependent cell-mediated cytotoxicity (R-ADCC).

55. A method of treating a myeloma in a human patient, comprising administering an antibody to the patient, wherein
the antibody binds SLAMF7 on the surface of a natural killer (NK) cell; and
an Fc portion of the antibody to an Fc receptor on a cell of the myeloma, said receptor being selected from the group consisting of CD16, CD32, and CD64; and
wherein killing of the myeloma is via reverse antibody dependent cell-mediated cytotoxicity (R-ADCC).

56. A method according to claim 1, wherein the cancer is leukemia, wherein the patient is a human patient, wherein the antigen is selected from the group consisting of NKp30, NKp44, and SLAMF-7, and the Fc receptor is selected from the group consisting of CD16, CD32, and CD64.
